(19) United States
(12) Patent Application Publication
(10) Pub. No.: US 2009/0266359 A1
(43) Pub. Date: Oct. 29, 2009

(54) Patient therapy equipment

(75) Inventor: Jonathan McNeill Flint, London (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Medical International Limited, London (GB)

(21) Appl. No.: 12/385,738

(22) Filed: Apr. 17, 2009

(30) Foreign Application Priority Data
Apr. 26, 2008 (GB) 0807641.6

(51) Int. Cl. A61M 16/00

(52) U.S. Cl. 128/204.18

(57) ABSTRACT

Sleep apnoea treatment equipment has a housing containing a blower connected to the housing outlet via a duct. A length of flexible breathing tubing connects the housing outlet with a face mask worn by the patient so that he receives a flow of air at elevated pressure. An acoustic sensor is mounted in the duct close to the blower and supplies an output to a processor. The processor supplies an output to a loudspeaker or other acoustic source mounted downstream of the sensor, between it and the outlet. The processor drives the source to produce anti-noise in anti-phase with the blower-generated noise so as to reduce the noise level outside the equipment.

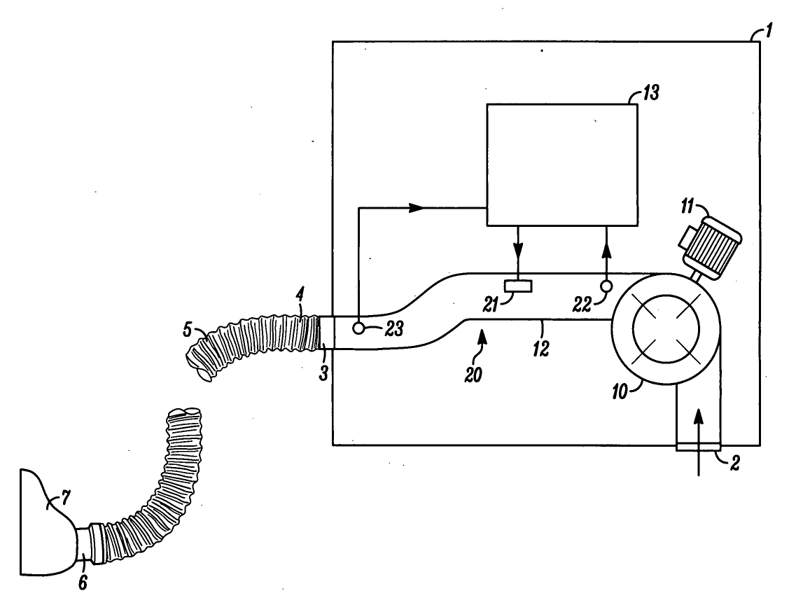

### BACKGROUND OF THE INVENTION

This invention relates to patient therapy equipment.

The invention is more particularly concerned with equipment for delivering breathing gas to a patient at elevated pressure, such as to relieve breathing problems.

CPAP or continuous positive airway pressure equipment is used by patients suffering from sleep apnoea problems. The equipment includes some form of electrically-operated blower connected by tubing to a patient interface, such as a face mask. Some equipment may operate in a bi-level mode where the applied pressure reduces when the patient exhales. CPAP equipment is available from various manufacturers, such as Respironics, Resmed, Hoffrichter and Fisher & Paykel. The equipment is usually used by the patient at home, while the patient is in bed, to relieve sleep problems, so it is very important that the noise produced by the equipment is as low as possible, both to reduce the risk of disturbing the patient and his or her partner. Manufacturers take great care to reduce the noise produced by using low-noise motors, careful acoustic design and by the incorporation of sound-absorbing materials. In this way, the best modern equipment can achieve noise levels of around 25 dbA. Examples of arrangements for reducing noise in such equipment are described in WO9922793, EP1318307, EP1643131, U.S. Pat. No. 6,615,831, U.S. Pat. No. 7,134,434 and WO2007/076570.

### BRIEF SUMMARY OF THE PRESENT INVENTION

It is an object of the present invention to provide alternative patient therapy equipment.

According to one aspect of the present invention there is provided patient therapy equipment including a housing containing an air-moving device arranged to provide a flow of air at above atmospheric pressure, an outlet of the housing, and a length of breathing gas tubing connected at one end with the housing outlet and at its other end with a patient breathing interface, the equipment including anti-noise generating means arranged to generate anti-noise effective to cancel at least in part the noise produced by parts of the equipment.

The anti-noise generating means preferably includes an acoustic sensor and an acoustic source, the sensor being mounted between the source and a noise-generating component of the equipment. The equipment preferably includes an additional acoustic sensor mounted downstream of the acoustic source and arranged to provide an electrical output indicative of the resulting noise level after addition of the anti-noise. The equipment may be arranged to adjust the phase and amplitude of the output of the acoustic source in accordance with the output of the additional sensor. The sensor and source may be mounted along a flow path between the air-moving device and the outlet. The air-moving device preferably includes a blower driven by a motor. The patient therapy equipment is preferably for use in the treatment of sleep apnoea, the patient breathing interface including a face mask.

According to another aspect of the present invention there is provided a method of reducing noise in patient therapy equipment of the kind arranged to deliver a flow of breathing gas to a patient's airways, including the steps of sensing noise produced by the equipment and generating anti-noise in anti-phase with the noise so as to reduce the. overall perceived noise level.

The method preferably includes the additional steps of sensing the resulting noise produced after addition of the anti-noise and adjusting the phase and amplitude of the anti-noise in accordance with the sensed resulting noise in a feedback manner to reduce the resulting sensed noise.

### BRIEF DESCRIPTION OF THE DRAWING

Equipment according to the present invention will now be described, by way of example, with reference to the accompanying drawing, which shows the equipment schematically.

### DETAILED DESCRIPTION OF THE PREFERRED EMBODIMENT

The equipment includes a housing 1 with an air inlet 2 open to atmosphere and an outlet 3 for pressurized air. The outlet 3 is connected to one end 4 of flexible breathing gas tubing 5, the other end of which is connected to the inlet 6 of a conventional face mask 7. It will be appreciated that the tubing 5 could, instead be connected to other forms of patient interface, such as nasal masks or breathing tubes. The housing 1 is arranged to be supported on a table or the like at the side of a patient's bed.

The housing 1 contains a conventional turbine blower 10, driven by an electrical motor 11 and operable to drive air from the inlet 2 to the outlet 3 via a gas-flow path or duct 12 within the housing. Operation of the motor 1 1 is controlled by a processor or control unit 13, which also controls various conventional safety functions. As so far described, the equipment is entirely conventional and it will be appreciated could operate as a simple CPAP device or have a bi-level or other function. The equipment may be designed to reduce noise in conventional ways, such as by the use of mufflers, sound-absorbing linings, low-noise motor or the like.

The equipment differs from conventional equipment in that it also includes an arrangement 20 to generate anti-noise to cancel out noise produced by noise-producing parts of the equipment, namely the motor, blower and air-flow noise along the duct 12. This anti-noise arrangement 20 includes an acoustic source 21, such as a loudspeaker or other sounder operable to produce sound within the frequency range of the noise produced by the equipment. The source 21 is mounted at a location along the duct 12 and is spaced towards the outlet 3 away from the blower 10. The source 21 is driven by an output from the control unit 13. The anti-noise arrangement 20 also includes a first acoustic sensor 22, such as a microphone or accelerometer mounted along the duct 12 close to the blower 10, that is, upstream of the source 21. The electrical output of the sensor 22 is connected to the processor 13. The arrangement 20 is completed by an optional second acoustic sensor 23, similar to the first sensor 22. This is mounted close to the outlet 3, that is, downstream of the source 21.

The anti-noise arrangement 20 operates in a manner well known in other applications. The first sensor 22 supplies an electrical noise output signal to the control unit 13, which generates an output to drive the source 21. The output to the source 21 is identical to the sensed noise provided by the sensor 22 but in anti-phase with it, that is, in anti-phase with the noise by the time it has reached the location of the source. The control unit 13 may simply amplify the output of the sensor 22 and use this to drive the source 21 after having appropriately adjusted the delay in the signal supplied to the source so as to achieve the desired phasing. If the phase and amplitude of the anti-noise produced by the source 21 are correctly set, it will interfere with and cancel the majority of the noise present in the duct 12. The second, downstream sensor 23 provides an electrical output representative of the resulting noise level at the outlet 3 after addition of the anti-noise. This is used by the control unit 13 in a feedback manner to adjust the amplitude and phase of the anti-noise signal produced by the source 21 so as to minimize the resulting noise at the outlet 3 and hence the overall noise level perceived by the user.

The arrangement described above is organized primarily to reduce noise in the duct 12. The acoustic sensor could, however, instead be mounted adjacent the motor 11 or the blower bearings and the sound source arranged primarily to reduce the resulting noise from these components. The equipment could include several different anti-noise arrangements arranged to reduce noise from different sources at different locations within the housing. The anti-noise source could be mounted outside the duct in the housing or could be external of the housing.

Instead of using an acoustic sensor, it might be possible to derive an indication of, for example, motor noise in some other way, such as from a speed sensor or by monitoring an electrical signal across the motor. In such an arrangement, the control unit could be arranged to generate the anti-noise signal such as from a sound synthesizer arranged, for example, to increase frequency as the detected motor speed increases.

The anti-noise arrangement of the present invention can be used in conjunction with conventional noise reducing features, such as insulation, damping, low-noise motor and the like, so as to reduce further the overall noise level of the equipment. Alternatively, the anti-noise arrangement could be used to enable, for example, a low-cost but higher-noise motor to be used, thereby reducing the overall cost of the equipment whilst still enabling low overall noise levels to be achieved.

What I claim is:

1. Patient therapy equipment comprising: a housing having an outlet, an air-moving device within the housing arranged to provide a flow of air at above atmospheric pressure, a length of breathing gas tubing connected at one end with the outlet of the housing and at an opposite end with a patient breathing interface, and an anti-noise generator arranged to generate anti-noise effective to cancel at least in part noise produced by the equipment.

2. Patient therapy equipment according to claim 1, wherein the anti-noise generator includes an acoustic sensor and an acoustic source, and wherein the acoustic sensor is mounted between the acoustic source and a part of the equipment that generates noise.

3. Patient therapy equipment according to claim 2 including an additional acoustic sensor mounted downstream of the acoustic source, wherein said additional acoustic sensor is arranged to provide an electrical output indicative of a resulting noise level after addition of the anti-noise.

4. Patient therapy equipment according to claim 3, wherein the equipment is arranged to adjust phase and amplitude of an output of the acoustic source in accordance with the output of the additional acoustic sensor.

5. Patient therapy equipment according to claim 2, wherein the acoustic sensor and acoustic source are mounted along a flow path between the air-moving device and the outlet.

6. Patient therapy equipment according to claim 1, wherein the air-moving device includes a blower and an electric motor arranged to drive the blower.

7. Patient therapy equipment for use in the treatment of sleep apnoea comprising: a housing having an outlet, an air-moving device within the housing arranged to provide a flow of air at above atmospheric pressure, a length of breathing gas tubing connected at one end with the outlet of the housing and at an opposite end with a face mask, and an anti-noise generator arranged to generate anti-noise effective to cancel at least in part noise produced by the equipment.

8. Patient therapy equipment for use in the treatment of sleep apnoea comprising: a housing having an outlet, an electrically-driven air blower within the housing arranged to provide a flow of air at above atmospheric pressure, a duct extending within the housing between the blower and the housing outlet, a processor, an acoustic sensor mounted along the duct to sense noise within the duct, a connection between said sensor and said processor, an acoustic source mounted along the duct between said acoustic sensor and said outlet, a connection between said acoustic source and said processor, and a length of breathing gas tubing connected at one end with the outlet of the housing and at an opposite end with a face mask, and wherein said processor is arranged in response to an output of said acoustic sensor to cause said acoustic source to generate anti-noise in effective to reduce noise perceived outside said housing.

9. A method of reducing noise in patient therapy equipment of the kind arranged to deliver a flow of breathing gas to a patient's airways, comprising the steps of sensing noise produced by the equipment and generating anti-noise in anti-phase with the noise so as to reduce the overall perceived noise level.

10. A method according to claim 9, wherein the method includes the additional step of sensing resulting noise produced after addition of the anti-noise and adjusting phase and amplitude of the anti-noise in accordance with the sensed resulting noise in a feedback manner to reduce the resulting sensed noise.
